(19) United States
(12) Patent Application Publication
(10) Pub. No.: US 2024/0428770 A1
(43) Pub. Date: Dec. 26, 2024

(54) SOUND MASKING APPARATUS AND METHOD

(71) Applicants: HYUNDAI MOTOR COMPANY, Seoul (KR); KIA CORPORATION, Seoul (KR)

(72) Inventors: Ki Chang Kim, Suwon-si (KR); Dong Chul Park, Anyang-si (KR)

(73) Assignees: HYUNDAI MOTOR COMPANY, Seoul (KR); KIA CORPORATION, Seoul (KR)

(21) Appl. No.: 18/387,347

(22) Filed: Nov. 6, 2023

(30) Foreign Application Priority Data
Jun. 20, 2023 (KR) 10-2023-0079185

(51) Int. Cl. G10K 11/175; A61B 5/00; A61M 21/02; G06V 40/10

(52) U.S. Cl. CPC G10K 11/1752; A61B 5/4809; A61M 21/02; G06V 40/15

(57) ABSTRACT

A sound masking apparatus and method are provided. The sound masking apparatus includes a sensing device that measures image information and biometric information of a passenger in a vehicle. The apparatus also includes a controller connected with the sensing device. The controller determines a sleep state of the passenger based on the biometric information and determines whether the passenger is disturbed in sleep based on the image information in the sleep state of the passenger. The controller measures noise in the vehicle in response to determining that the passenger is disturbed in sleep and determines whether there is a need for sound masking based on the measured noise. The controller performs the sound masking based on the sleep state in response to determining that there is the need for the sound masking.

### CROSS-REFERENCE TO RELATED APPLICATION

This application claims the benefit of and priority to Korean Patent Application No. 10-2023-0079185, filed in the Korean Intellectual Property Office on Jun. 20, 2023, the entire contents of which are incorporated herein by reference.

### TECHNICAL FIELD

The present disclosure relates to a sound masking apparatus and method.

### BACKGROUND

Various technologies may be utilized in an autonomous vehicle to provide ease and comfort in the vehicle as well as safety and driving performance. A noise reduction device may be provided as one of such technologies. The noise reduction device may analyze noise generated while the vehicle is traveling in real time and may generate an opposite phase sound wave for attenuating the noise to reduce the noise.

### SUMMARY

Aspects of the present disclosure provide a sound masking apparatus and method for determining a sleep state of a user to guide the user to sleep and for masking a sleep disturbance sound.

Other aspects of the present disclosure provide a sound masking apparatus and method for determining a sleep stage and sleep quality based on image information and/or biometric information and for playing a masking sound (or a masking sound source) based on the determined sleep stage and/or the determined sleep quality.

The technical problems to be solved by the present disclosure are not limited to the aforementioned problems. Other technical problems not mentioned herein should be more clearly understood from the following description by those having ordinary skill in the art to which the present disclosure pertains.

According to an aspect of the present disclosure, a sound masking apparatus is provided. The sound masking apparatus may include a sensing device that measures image information and biometric information of a passenger in a vehicle. The sound masking device may also include a controller connected with the sensing device. The controller may determine a sleep state of the passenger based on the biometric information. The controller may also determine whether the passenger is disturbed in sleep in the sleep state of the passenger based on the image information. The controller may measure noise in the vehicle in response to determining that the passenger is disturbed in sleep. The controller may determine whether there is a need for sound masking based on the measured noise. The controller may perform the sound masking based on the sleep state in response to determining that there is the need for the sound masking.

The controller may analyze a pattern of an electroencephalogram (EEG) signal measured by an EEG measurement device. The controller may determine the sleep state of the passenger based on the analyzed pattern of the EEG signal.

The controller may monitor a change in a body organ of the passenger and body movement of the passenger by analyzing the image information. The controller may also determine whether the passenger is disturbed in sleep based on at least one of the change in a body organ of the passenger or a body movement pattern, or any combination thereof.

The controller may determine that the passenger is disturbed in sleep when at least one of an eye blinking duration of the passenger is less than or equal to a predetermined reference time or a body movement pattern is a rapid eye movement (REM) sleep behavior disorder pattern, or any combination thereof.

The controller may compare a magnitude of the measured noise with a predetermined reference value. The controller may also determine that there is the need for the sound masking when the magnitude of the measured noise is greater than or equal to the predetermined reference value.

The controller may select white noise as a masking sound source when the sleep state switches from a sleep boundary stage to a sleep stage. The controller may also play and output the selected white noise.

The controller may randomly play the selected white noise.

The controller may select pink noise as a masking sound source when the sleep state switches from a sleep stage to a deep sleep stage. The controller may also play and output the selected pink noise.

The controller may control a magnitude of the pink noise based on a magnitude of the measured noise.

The controller may select and play a masking sound source based on a purpose of use of the sound masking.

According to another aspect of the present disclosure, a sound masking method is provided. The sound masking method may include determining a sleep state of a passenger in a vehicle based on biometric information of the passenger. The biometric information may be measured by a sensing device. The sound masking method may also include determining whether the passenger is disturbed in sleep based on image information of the passenger. The image information may be obtained by the sensing device in the sleep state of the passenger. The sound masking method may further include measuring noise in the vehicle in response to determining that the passenger is disturbed in sleep. The sound masking method may additionally include determining whether there is a need for sound masking based on the measured noise. The sound masking method may further still include performing the sound masking based on the sleep state in response to determining that there is the need for the sound masking.

Determining the sleep state of the passenger may include analyzing a pattern of an EEG signal measured by an EEG measurement device and determining the sleep state of the passenger based on the analyzed pattern of the EEG signal.

Determining whether the passenger is disturbed in sleep may include monitoring a change in a body organ of the passenger and body movement of the passenger by analyzing the image information and may include determining whether the passenger is disturbed in sleep based on at least one of the change in body organ of the passenger or a body movement pattern, or any combination thereof.

Determining whether the passenger is disturbed in sleep may include determining that the passenger is disturbed in sleep when at least one of an eye blinking duration of the passenger is less than or equal to a predetermined reference time or a body movement pattern is a REM sleep behavior disorder pattern, or any combination thereof.

Determining whether there is the need for the sound masking may include comparing a magnitude of the measured noise with a predetermined reference value and determining that there is the need for the sound masking when the magnitude of the measured noise is greater than or equal to the predetermined reference value.

Performing the sound masking may include selecting white noise as a masking sound source when the sleep state switches from a sleep boundary stage to a sleep stage and may include playing and outputting the selected white noise.

The playing and outputting of the selected white noise may include randomly playing the selected white noise.

Performing the sound masking may include selecting pink noise as a masking sound source when the sleep state switches from a sleep stage to a deep sleep stage and may include playing and outputting the selected pink noise.

Playing and outputting the selected pink noise may include controlling a magnitude of the pink noise based on a magnitude of the measured noise.

Performing the sound masking may include selecting and playing a masking sound source based on a purpose of use of the sound masking.

### BRIEF DESCRIPTION OF THE DRAWINGS

The above and other objects, features and advantages of the present disclosure should be more apparent from the following detailed description taken in conjunction with the accompanying drawings, in which:

FIG. 1 is a block diagram illustrating a configuration of a sound masking apparatus, according to embodiments of the present disclosure;

FIG. 2 is a drawing for describing a process of determining a sleep state, according to embodiments of the present disclosure;

FIG. 3 is a drawing for describing a process of determining a sleep stage, according to embodiments of the present disclosure;

FIG. 4 is a drawing for describing a method for determining sleep disturbance, according to embodiments of the present disclosure;

FIG. 5 is drawing for describing a process of determining whether there is a need for sound masking, according to embodiments of the present disclosure;

FIG. 6 is a drawing illustrating the result of analyzing a white noise contribution, according to embodiments of the present disclosure;

FIG. 7 is a drawing illustrating the result of analyzing a pink noise contribution, according to embodiments of the present disclosure;

FIG. 8 is a drawing for describing a process of applying a cocktail party effect, according to embodiments of the present disclosure;

FIG. 9 is a drawing for describing a sound masking concept, according to embodiments of the present disclosure;

FIG. 10 is a drawing for describing a sound masking algorithm, according to embodiments of the present disclosure;

FIG. 11 is a drawing for describing passive sound masking, according to embodiments of the present disclosure;

FIG. 12 is a drawing for describing active sound masking, according to embodiments of the present disclosure;

FIG. 13 is a flowchart illustrating a sound masking method, according to embodiments of the present disclosure; and

FIG. 14 is a block diagram illustrating a configuration of a sound masking system, according to embodiments of the present disclosure.

### DETAILED DESCRIPTION

Hereinafter, embodiments of the present disclosure are described in detail with reference to the accompanying drawings. In the drawings, the same reference numerals are used throughout to designate the same or equivalent elements. In addition, a detailed description of well-known features or functions has been omitted in order not to unnecessarily obscure the gist of the present disclosure. When a component, device, element, or the like of the present disclosure is described as having a purpose or performing an operation, function, or the like, the component, device, or element: should be considered herein as being “configured to” meet that purpose or perform that operation or function.

In describing the components of the embodiment according to the present disclosure, terms such as first, second, “A”, “B”, (a), (b), and the like may be used. These terms are only used to distinguish one element from another element. These terms do not limit the corresponding elements irrespective of the order or priority of the corresponding elements. Furthermore, unless otherwise defined, all terms, including technical and scientific terms, used herein should be interpreted as is customary in the art to which the present disclosure pertains. It should be understood that terms used herein should be interpreted as having a meaning that is consistent with their meaning in the context of this disclosure and the relevant art and should not be interpreted in an idealized or overly formal sense unless expressly so defined herein.

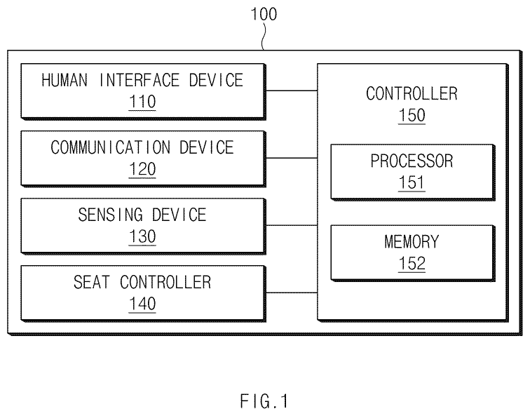

FIG. 1 is a block diagram illustrating a configuration of a sound masking apparatus, according to embodiments of the present disclosure.

A sound masking apparatus 100 may be mounted on a vehicle equipped with an autonomous driving function. The autonomous driving function may be of Level 3 or higher among Automation Levels 0 to 5 defined by the Society of Automotive Engineers (SAE). Level 3 (partial automation) is a level where the system takes charge of driving in a road or route section in a specific condition, for example, a highway, and where a driver intervenes only in case of danger.

Referring to FIG. 1, the sound masking apparatus 100 may include a human interface device 110, a communication device 120, a sensing device 130, a seat controller 140, and a controller 150, which may be connected over an in-vehicle or inter-vehicle network. The inter-vehicle network may be implemented as a controller area network (CAN), a media oriented systems transport (MOST) network, a local interconnect network (LIN), an Ethernet, X-by-Wire (Flexray), and/or the like.

The human interface device 110 may be a device that facilitates interaction between the sound masking apparatus 100 and a user (e.g., a driver or a passenger). The human interface device 110 may include an input device (e.g., a keyboard, a touch pad, a microphone, a touch screen, and/or the like) for generating data according to manipulation of the user, an output device (e.g., a display, a speaker, a vibration generation device, and/or the like) for outputting information according to an operation of the sound masking apparatus 100, and/or the like. Such a human interface device 110 may be implemented as an audio, video, navigation (AVN) terminal, an in-vehicle infotainment terminal, a telematics terminal, a portable speaker, and/or the like.

The human interface device 110 may include a function on/off button capable of turning on or off a sleep therapy function. The human interface device 110 may also include an on/off button capable of turning on or off a sound masking function. The function on/off buttons may be implemented as hardware buttons and/or software buttons.

The communication device 120 may enable communication between the sound masking apparatus 100 and an external electronic device (e.g., a server, an electronic control unit (ECU), a user device, a smartphone, or the like). The communication device 120 may include a wireless communication circuit (e.g., a cellular communication circuit, a wireless-fidelity (Wi-Fi) communication circuit, a short-range wireless communication circuit, and/or a global navigation satellite system (GNSS) communication circuit) and/or a wired communication circuit (e.g., a local area network (LAN) communication circuit and/or a power line communication circuit). The communicate device 120 may receive a masking sound source transmitted from the external electronic device.

The sensing device 130 may measure image information and biometric information of a passenger in the vehicle using a camera and/or a biometric signal measurement device (e.g., an electroencephalogram (EEG) measurement device or an electrocardiogram (ECG) measurement device). The biometric information may include EEG, a heart rate, and/or the like.

The sensing device 130 may measure interior noise of the vehicle using a microphone. At least one microphone may be installed in the vehicle. For example, the microphones may be installed in a dashboard and the periphery of an interior light at the rear seat, respectively.

The sensing device 130 may obtain driving information using sensors, an electronic control unit, and/or the like mounted on the vehicle. The sensors may include a wheel speed sensor, an inertial measurement unit (IMU), an image sensor, radio detecting and ranging (RADAR), light detection and ranging (LiDAR), a steering angle sensor, and/or the like. The driving information may include a vehicle speed, a road curvature, lane departure or not, a steering angle, and/or the like.

The seat controller 140 may control a plurality of vibrators installed in the vehicle seat. When a sleep therapy function is turned on, the seat controller 140 may control the plurality of vibrators based on a predetermined vibration frequency, a predetermined vibration pattern, a predetermined vibration waveform, and/or the like. In other words, the seat controller 140 may control the plurality of vibrators to generate seat vibration.

The controller 150 may control the overall operation of the sound masking apparatus 100. The controller 150 may include a processor 151 and a memory 152. The processor 151 may be implemented as at least one of an application specific integrated circuit (ASIC), a digital signal processor (DSP), a programmable logic device (PLD), a field programmable gate array (FPGA), a central processing unit (CPU), a microcontroller, or a microprocessor, or a combination thereof. The memory 152 may be a non-transitory storage medium that stores instructions executed by the processor 151. The memory 152 may be implemented at least one of storage media, such as a flash memory, a hard disk, a solid state disk (SSD), a random access memory (RAM), a static RAM (SRAM), a read only memory (ROM), a programmable ROM (PROM), an electrically erasable and programmable ROM (EEPROM), or an erasable and programmable ROM (EPROM). The memory 152 may store a sound masking algorithm, sound masking control logic, and/or the like.

In response to receiving a command to turn on a deep sleep therapy function from the human interface device 110, the controller 150 may execute the deep sleep therapy function. When the deep sleep therapy function is executed, the controller 150 may control the seat controller 140 to generate vibration for guiding a user to have a deep sleep on the seat. Furthermore, the controller 150 may play a sleep guidance sound to output the sleep guidance sound to the outside through the human interface device 110.

The controller 150 may monitor a facial expression, a sitting posture, and/or EEG (or an EEG signal) of a passenger using the sensing device 130. The controller 150 may monitor a facial expression, a sitting posture (or tossing and turning or body movement), an EEG pattern of the passenger by means of a camera and/or a biometric signal measurement device. The controller 150 may monitor a change in body organ (e.g., eyes), for example, gaze and/or eye blinking of the passenger. The controller 150 may determine a sleep state as a sleep stage, when the eye blinking duration of the passenger is greater than or equal to, for example, 800 milliseconds (ms). The controller 150 may determine the sleep state as a sleep boundary stage, when the eye blinking duration of the passenger is greater than, for example, 400 ms and is less than, for example, 800 ms. Furthermore, the controller 150 may determine the sleep state as a sleep disturbance stage (or a sleep disturbance state), when the eye blinking duration of the passenger is less than or equal to, for example, 400 ms. The controller 150 may monitor a change in sitting posture (e.g., the amount of behavior) of the passenger. When periodic motion of the passenger is recognized, the controller 150 may determine that the passenger is in normal sleep. When a rapid eye movement (REM) sleep behavior disorder (e.g., dream behavior) pattern where the passenger flinches and makes large motion is recognized, the controller 150 may determine that the passenger is disturbed in sleep. The controller 150 may analyze a sleep cycle through EEG measurement. The controller 150 may determine normal sleep or sleep disturbance based on the result of analyzing the sleep cycle.

The controller 150 may determine whether the passenger is disturbed in sleep based on the image information obtained by the camera and/or the biometric signal measured by the biometric signal measurement device.

When it is determined that the passenger is disturbed in sleep, the controller 150 may measure noise in the vehicle using the microphone. When it is determined that the passenger is not disturbed in sleep, the controller 150 may determine that the sleep state is the normal sleep (or the normal sleep state).

The controller 150 may determine whether there is a need for sound masking based on a magnitude of the noise measured by the microphone. The controller 150 may compare the magnitude of the measured noise with a predetermined reference value and may determine whether there is a need for sound masking based on the result of the comparison.

When it is determined that there is a need for sound masking, the controller 150 may perform the sound masking. The controller 150 may determine a masking sound (or a masking sound source) based on the sleep state and/or the purpose of use (e.g., reduction of noise between floors, stress relief, concentration on work, or the like). At least one of white noise, pink noise, or brown noise, or any combination thereof, may be used as the masking sound. White noise may be noise in electronic equipment produced by electron free motion. White noise generally has a uniform frequency spectrum. White noise may be the sound of rain, the sound of waves, the sound of mountain temple bells, the sound of birds, or the like. Pink noise may be noise that has the same amount of energy at every octave. Pink noise may be used as a sound source for measuring and testing an acoustic characteristic. Pink noise may be the sound of a forest, the sound of a stream, the sound of wind, the sound of grass bugs, or the like. Brown noise may be a sound in which a high frequency domain is fully removed from the white noise. As a result, brown noise may sound more comfortable in the lower register. Brown noise may be the distant sound of rushing water, the sound of thunder, or the like.

When it is determined that there is no need for sound masking, the controller 150 may determine the measured noise as acceptable noise. In other words, when it is determined that there is no need for sound masking, the controller 150 may determine that the measured noise is acceptable.

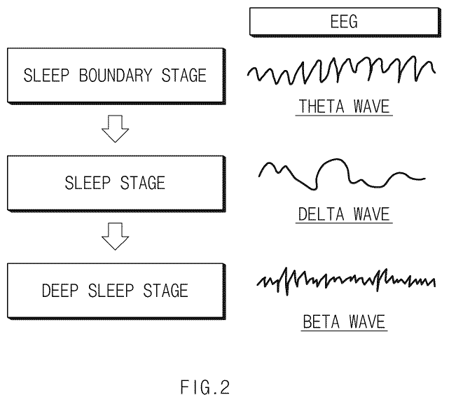

FIG. 2 is a drawing for describing a process of determining a sleep state, according to embodiments of the present disclosure.

Referring to FIG. 2, the controller 150 of the sound masking apparatus 100 of FIG. 1 may measure an EEG signal (or EEG) using an EEG measurement device. The controller 150 may analyze a pattern of the measured EEG signal and may determine a sleep state of a user (e.g., a passenger, a driver, or the like) based on the pattern.

When the measured EEG signal is a theta wave, the controller 150 may determine the sleep state (or a sleep stage) as a sleep boundary stage (or a first sleep stage or a sleep entry stage). The controller 150 may determine the sleep state as a sleep stage (or a second sleep stage or a light sleep stage) when the measured EEG signal is a delta wave. The controller 150 may determine the sleep state as a deep sleep stage (or a third sleep stage) when the measured EEG signal is a beta wave.

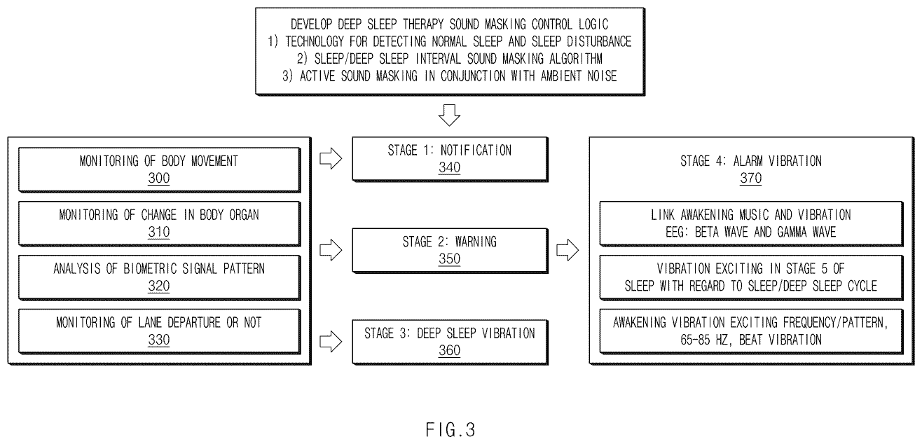

FIG. 3 is a drawing for describing a process of determining a sleep stage, according to embodiments of the present disclosure.

Referring to FIG. 3, the controller 150 of the sound masking apparatus 100 of FIG. 1 may perform monitoring 300 of body movement of a passenger by means of a camera. For example, the controller 150 may analyze image information obtained by the camera. The controller 150 may detect tossing and turning of the passenger, a change in sitting posture of the passenger, and/or the like based on the image information.

The controller 150 may perform monitoring 310 of a change in body organ of the passenger by means of the camera. The controller 150 may detect eye blinking of the passenger, a change in gaze of the passenger, and/or the like from image information obtained by the camera.

The controller 150 may perform an analysis 320 of a biometric signal pattern using a biometric signal measurement device. The controller 150 may measure an EEG signal using an EEG measurement device and may analyze a pattern of the measured EEG signal.

The controller 150 may perform monitoring 330 of lane departure by means of a lane departure warning system while the vehicle is traveling.

The controller 150 may determine (or recognize) a sleep stage based on the result of monitoring the body movement of the body and/or the change in body organ, the result of analyzing the biometric signal pattern and/or the result of monitoring the lane departure or not, or the like. The sleep stage may be classified into three stages or four stages, for example.

The controller 150 may apply a sleep induction method according to the sleep stage.

When the sleep stage is determined as stage 1, the controller 150 may continuously output a notification 340 of the necessity of sleep using a voice Interaction technology through a display, a speaker, and/or the like.

When the sleep stage is determined as stage 2, the controller 150 may guide a driver to a rest area, a sleep shelter, and/or the like in conjunction with a navigation terminal. The controller 150 may also output a warning 350 indicating that there is a possibility of drowsy driving.

When the sleep stage is determined as stage 3, the controller 150 may generate deep sleep vibration 360 on the seat using the sleep induction when the vehicle is turned off technology.

When the sleep stage is determined as stage 4, the controller 150 may generate alarm vibration 370 based on a frequency that wakes the driver up pleasantly and a seat vibration pattern on the seat. As an example, when EEG is a beta wave and a gamma wave, the controller 150 may link and output awakening music and vibration. As another example, the controller 150 may output vibration exciting in stage 5 of sleep with regard to the sleep and deep sleep cycle. As another example, when the awakening vibration exciting frequency is 65 Hz to 85 Hz, the controller 150 may output an awakening vibration exciting pattern as beat vibration.

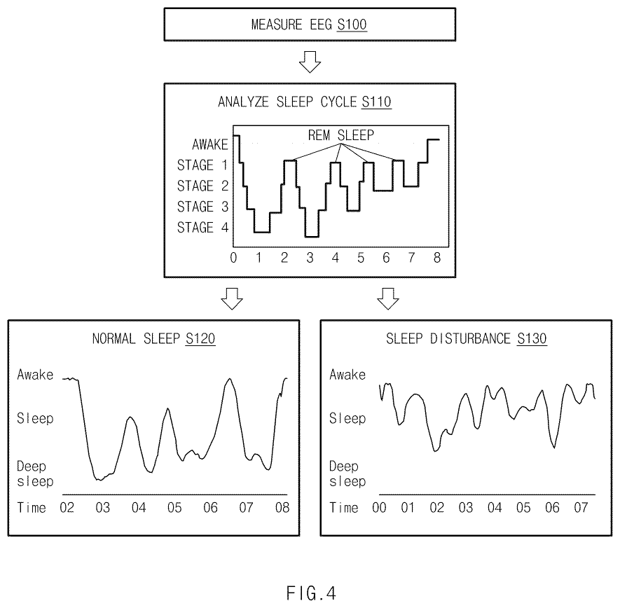

FIG. 4 is a drawing for describing a method for determining sleep disturbance, according to embodiments of the present disclosure.

Referring to FIG. 4, in an operation S100, the controller 150 of the masking apparatus 100 of FIG. 1 may measure an EEG signal by means of the sensing device 130 of FIG. 1. For example, the controller 150 may measure an EEG signal of a passenger by means of an EEG measurement device.

In an operation S110, the controller 150 may analyze a sleep cycle based on the measured EEG signal. The controller 150 may analyze the measured EEG signal. The controller 150 may determine a one-time sleep cycle (e.g., 90 minutes) composed of rapid eye movement (REM) sleep and non-REM sleep based on the EEG signal.

When the analyzed sleep cycle is a normal sleep cycle (or a pleasant sleep cycle), the controller 150 may, in an operation S120, determine the sleep state as normal sleep. When the sleep cycle is 90 minutes, the controller 150 may determine that the sleep cycle is the normal sleep cycle.

When the analyzed sleep cycle is a sleepless cycle (or a sleepless cycle incapable of sleeping deeply), the controller 150 may, in an operation S130, determine the sleep state as sleep disturbance. For example, when the sleep cycle is 30 minutes, the controller 150 may determine that the sleep cycle is a sleepless cycle.

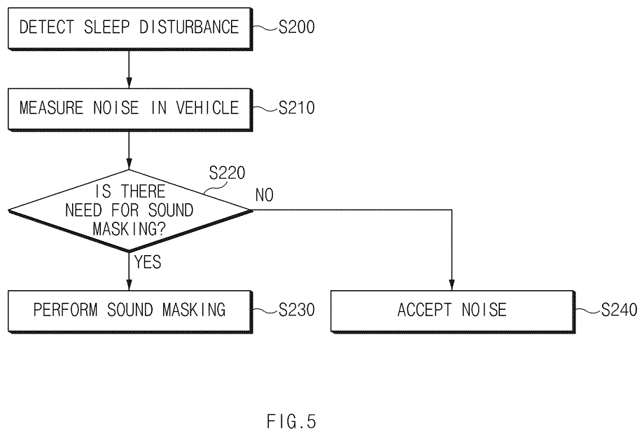

FIG. 5 is drawing for describing a process of determining whether there is a need for sound masking, according to embodiments of the present disclosure.

In an operation S200, the controller 150 of the sound masking apparatus 100 of FIG. 1 may detect sleep disturbance by means of the sensing device 130 of FIG. 1. The controller 150 may analyze image information received from a camera and may recognize whether there is sleep disturbance based on the image information. For example, when the eye blinking duration of a passenger is less than or equal to a reference time (e.g., 400 ms) and/or when a REM sleep behavior disorder pattern (e.g., tossing and turning) is detected, the controller 150 determine that the passenger is disturbed in sleep (i.e., in a sleep disturbance state).

When the sleep disturbance is detected, the controller 150 may, in an operation S210, measure noise in a vehicle using a microphone. The controller 150 may measure a magnitude of noise which disturbs the sleep of the passenger.

In an operation S220, the controller 150 may determine whether there is a need for sound masking based on the magnitude of the measured noise. The controller 150 may identify whether the magnitude of the measured noise is greater than or equal to a predetermined reference value (e.g., 66 decibels (dB)). When the magnitude of the measured noise is greater than or equal to the predetermined reference value, the controller 150 may determine that there is a need for sound masking. On the other hand, when the magnitude of the measured noise is less than the predetermined reference value, the controller 150 may determine that there is no need for sound masking.

When it is determined that there is a need for sound masking, the controller 150 may, in an operation S230, perform the sound masking. The controller 150 may output white noise or pink noise using a sound masking algorithm specialized for a low frequency. The controller 150 may play random noise-based white noise in a sleep interval. The controller 150 may play pink noise for emphasizing a mid-low sound in a deep sleep interval. The played noise may be output through a speaker. The controller 150 may randomly play the masking sound not familiar to the brain and ears, thus implementing a cocktail party effect. Furthermore, the controller 150 may perform active sound masking in conjunction with noise around a vehicle and an office.

When it is determined that there is no need for sound masking, the controller 150 may, in an operation S240, determine that noise in the vehicle is an acceptable degree.

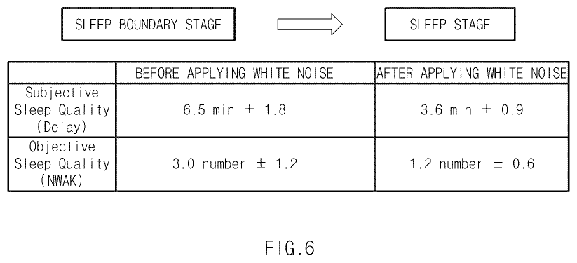

FIG. 6 is a drawing illustrating the result of analyzing a white noise contribution, according to embodiments of the present disclosure.

A sound masking apparatus 100 of FIG. 1 may analyze sleep quality according to there is white noise in a situation where a sleep state switches from a sleep boundary stage to a sleep stage.

The sound masking apparatus 100 may monitor eye blinking of a passenger by means of a camera. When the eye blinking duration of the passenger is greater than, for example, 400 ms and is less than, for example, 800 ms, the controller 150 may determine the sleep state as the sleep boundary stage. When the eye blinking duration of the is greater than or equal to, for example, 800 ms, the controller 150 may determine the sleep state as the sleep stage. When the eye blinking duration is less than or equal to, for example, 400 ms, the controller 150 may determine the sleep state as a sleep disturbance stage.

The controller 150 may monitor the number of times that the passenger tosses and turns his or her body and a pattern where the passenger tosses and turns his or her body, by means of the camera. When the number of times that the passenger tosses and turns his or her body is less than or equal to, for example, 2 times and when the pattern where the passenger tosses and turns his or her body is periodic movement, the controller 150 may determine the sleep state as normal sleep. When the number of times that the passenger tosses and turns his or her body is greater than or equal to, for example, 3 times and when the pattern where the passenger tosses and turns his or her body is a flinching REM sleep behavior disorder pattern, the controller 150 may determine the sleep state as sleep disturbance.

The controller 150 may monitor a biometric signal (e.g., an EEG signal) of the passenger using a biometric signal measurement device. The controller 150 may analyze a sleep cycle by means of the biometric signal of the passenger. When the sleep cycle is a pleasant sleep cycle, the controller 150 may determine the sleep state as normal sleep. When the sleep cycle is a sleepless cycle incapable of sleeping deeply, the controller 150 may determine the sleep state as sleep disturbance.

When it is determined that the sleep state is the sleep disturbance, the controller 150 may play and output a white noise sound source. A sound source such as the sound of waves, the sound of rain, or the sound of running water may be used as the white noise sound source. The controller 150 may randomly play white noise at intervals of a predetermined time (e.g., 10 minutes). For example, the controller 150 may measure a time (or a sleep delay or sleep latency) taken to switch from the sleep boundary stage to the sleep stage to analyze sleep quality. It may be identified that the sleep delay is, for example, 6.5 min±1.8 before applying the white noise, but that the sleep delay decreases to, for example, 3.6 min±0.9 after applying the white noise. Accordingly, it may be identified that playing the white noise in the situation where the sleep state switches from the sleep boundary stage to the sleep stage is effective in reducing the sleep delay.

Additionally, or alternatively, the controller 150 may measure the number of awakening (NWAK) (or the number of times that the body is tossed and turned) to analyze sleep quality. It may be identified that the NWAK is, for example, 3.0±1.2 before applying the white noise, but that the NWAK decreases to, for example, 1.2 number±0.6 after applying the white noise.

As such, when playing the white noise in the situation where the sleep state switches from the sleep boundary stage to the sleep stage, because the time taken to switch from the sleep boundary stage to the sleep stage is reduced and/or because the number of times that the passenger tosses and turns his or her body is also reduced, it may be seen that playing the white noise improves sleep quality.

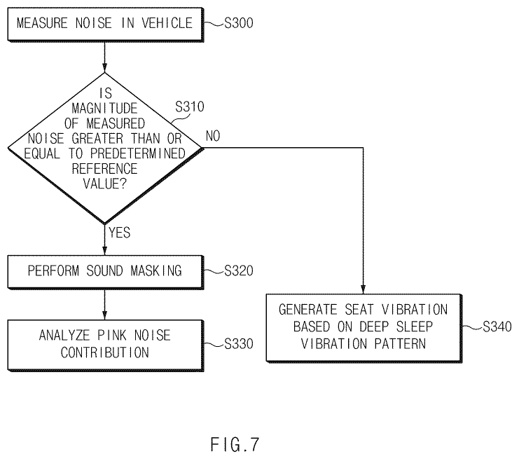

FIG. 7 is a drawing illustrating the result of analyzing a pink noise contribution, according to embodiments of the present disclosure.

In an operation S300, when sleep disturbance is detected in a situation where a sleep state switches from a sleep stage to a deep sleep stage, the controller 150 of the sound masking apparatus 100 of FIG. 1 may measure noise in a vehicle using a microphone. For example, when the eye blinking duration is less than or equal to a reference time (e.g., 400 ms) and/or when the pattern where the passenger tosses and turns his or her body is a REM sleep behavior disorder pattern, the controller 150 may determine that a passenger is disturbed in sleep. When it is determined that the passenger is disturbed in sleep, the controller 150 may measure noise that disturbs the sleep of the passenger in the vehicle.

In an operation S310, the controller 150 may determine whether a magnitude of the measured noise is greater than or equal to a predetermined reference value. For example, the controller 150 may identify whether the magnitude of the measured noise is greater than or equal to 66 dB.

When the magnitude of the measured noise is greater than or equal to the predetermined reference value, the controller 150 may, in an operation S320, perform sound masking using pink noise. The controller 150 may play and output the pink noise based on sound masking control logic.

In an operation S330, the controller 150 may analyze a pink noise contribution while performing the sound masking. The controller 150 may quantitatively analyze an effect of the pink noise on deep sleep at a sleep cycle to which the pink noise is applied.

For example, the controller 150 may analyze a time of restless (TRL), the number of restless (NRL), and/or a sleep depth (or a deep sleep degree or a depth) according to whether the pink noise is applied in the situation where the sleep state switches from the sleep stage to the deep sleep stage. The sleep depth may be defined as a difference between a maximum value and a minimum value on a vertical axis (or a y-axis) of the sleep cycle.

The result of quantitatively analyzing the time of restless (TRL), the number of restless (NRL), and the sleep depth according to whether the pink noise is applied in the situation where the sleep state switches from the sleep stage to the deep sleep stage, according to an embodiment, is as shown in Table 1 below.

TABLE 1

| | Before applying pink noise | After applying pink noise |
|---|---|---|
| TRL | 120 min ± 60 | 40 min ± 20 |
| NRL | 10 number ± 8 | 5 number ± 3 |
| depth | 8 ± 4 | 16 ± 4 |

As shown in Table 1, the time of restless (TRL) may be reduced, the number of restless (NRL) may be reduced, and/or the depth of sleep may be increased as pink noise is applied in the situation where the sleep state switches from the sleep stage to the deep sleep stage. When it is determined that the magnitude of the measured noise is not greater than or equal to the predetermined reference value, the controller 150 may, in an operation S340, control a seat controller 140 of FIG. 1 to generate seat vibration based on a deep sleep vibration pattern.

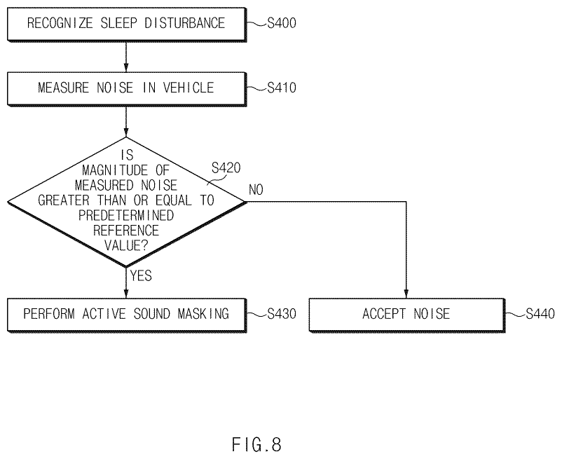

FIG. 8 is a drawing for describing a process of applying a cocktail party effect, according to embodiments of the present disclosure.

In an operation S400, the controller 150 of the sound masking apparatus 100 of FIG. 1 may analyze image information obtained by a camera and may recognize a state where a passenger is disturbed in sleep (i.e., sleep disturbance) based on the image information.

When the sleep disturbance is detected, the controller 150 may, in an operation S410, measure noise in a vehicle. The controller 150 may measure noise using a microphone installed in the vehicle.

In an operation S420, the controller 150 may determine whether the magnitude of the measured noise is greater than or equal to a predetermined reference value. When the magnitude of the measured noise is greater than or equal to the predetermined reference value, the controller 150 may determine that there is a need for sound masking. On the other hand, when the magnitude of the measured noise is not greater than or equal to the predetermined reference value, i.e., when the magnitude of the measured noise is less than the predetermined reference value, the controller 150 may determine that there is no need for sound masking.

When it is identified that the magnitude of the measured noise is greater than or equal to the predetermined reference value, the controller 150 may, in an operation S340, perform the sound masking. The controller 150 may select a masking sound source (or a masking sound) based on a sleep state. When the sleep state switches from a sleep boundary stage to a sleep stage, the controller 150 may select random noise-based white noise as the masking sound source. When the sleep state switches from the sleep stage to a deep sleep stage, the controller 150 may select pink noise, which is corrected and/or filtered with the same energy, as the masking sound source. The controller 150 may play and output the selected masking sound source through a speaker.

The controller 150 may randomly play white noise (e.g., the sound of running water or the sound of people speaking) and may perform passive sound masking. A cocktail party effect may be implemented through the passive sound masking.

The controller 150 may perform active sound masking to serve as a blanket for covering noise in the vehicle by playing pink noise (e.g., the sound of crashing waves, the sound of raindrops falling, or the like).

When the magnitude of the measured noise is not greater than or equal to the predetermined reference value, the controller 150 may, in an operation S440, determine that the noise in the vehicle is acceptable.

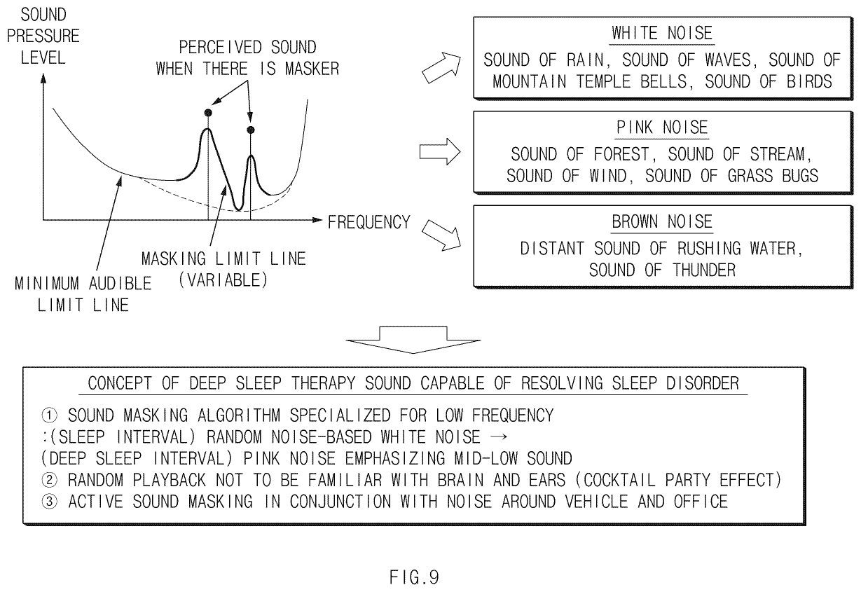

FIG. 9 is drawing for describing a sound masking concept, according to embodiments of the present disclosure.

Sound masking refers to a phenomenon in which one sound is drowned out by another sound. Disturbed large sound refers to a masker, and drowned small sound refers to a maskee. At least one of white noise, pink noise, or brown noise, or any combination thereof may be used as a masking sound source for the sound masking.

The sound masking apparatus 100 of FIG. 1 may play random noise-based white noise in a sleep interval and may play pink noise emphasizing a mid-low sound in a deep sleep interval, using a sound masking algorithm specialized for a low frequency.

The sound masking apparatus 100 may randomly play a masking sound source not familiar to the brain and ears, thus implementing a cocktail party effect.

Furthermore, the sound masking apparatus 100 may perform active sound masking in conjunction with noise around a vehicle and an office.

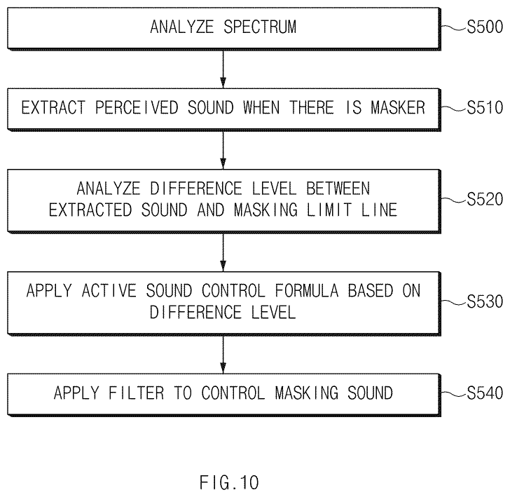

FIG. 10 is a drawing for describing a sound masking algorithm, according to embodiments of the present disclosure.

The sound masking algorithm may analyze spatial acoustic noise in a vehicle or a car park environment and may adjust a sound based on the spatial acoustic noise. The sound masking algorithm may prevent a sound from being too large or too small by means of active sound control. The sound masking algorithm may variably implement sound masking specialized for a low frequency.

In an operation S500, the controller 150 of the sound masking apparatus 100 of FIG. 1 may measure noise in vehicle and may perform a spectrum analysis of the measured noise.

In an operation S510, the controller 150 may extract a perceived sound when there is a masker by means of the spectrum analysis.

In an operation S520, the controller 150 may analyze a difference level between a sound pressure level of the extracted sound and a masking limit line.

In an operation S530, the controller 150 may apply an active sound control formula based on the analyzed difference level. The active sound control formula ASC (n) may be represented as Equation 1 below.

$$ASC ( n ) = Noise ( n ) - Mask ( n ) + margin \qquad Equation 1$$

The controller 150 may set a maximum value (or a maximum sound pressure level) and a minimum value (or a minimum sound pressure level) such that the masking sound is not too large or too small using ASC (n). The controller 150 may reflect a margin to variably apply the masking limit line when the masking sound increases and decreases.

In an operation S540, the controller 150 may apply a filter based on a sleep stage, REM sleep cycle information, or an alert and/or emergency information to control the masking sound. The controller 150 may apply the filter as in Equation 2 below, thus preventing the masking sound from being rapidly changed.

$$sound ( n ) = sound ( n - 1 ) + Filter \times ADC ( n ) \qquad Equation 2$$

The controller 150 may thus prevent the sense of difference due to a rapid change in sound pressure level of the masking sound.

The controller 150 may implement a cocktail party effect using the sound masking algorithm. The cocktail party effect stems from the fact that party attendees are selectively focused on, and are receptive to, conversations with an interlocutor despite being in a room with loud ambient noise. Selectively accepting only information meaningful to oneself regardless of the surrounding environment is called “selective perception” or “selective attention”. The cocktail party effect refers to a psychological phenomenon indicated by such selective perception or attention. The reason why the “cocktail party effect” occurs is that no matter how many different voices come into the ears, the human brain may pick and processes only one of them.

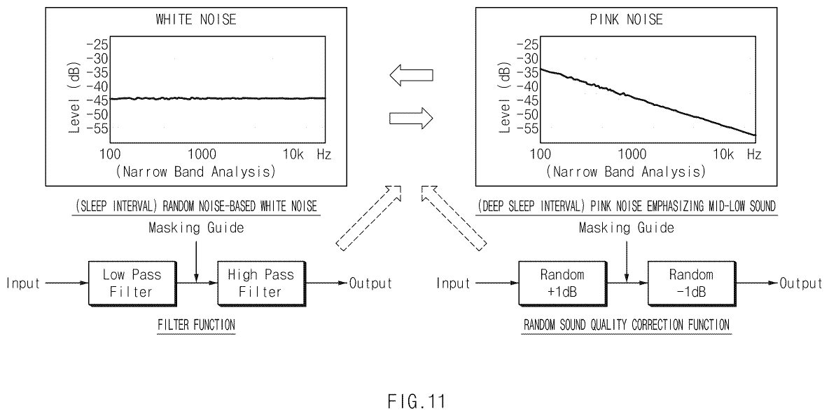

FIG. 11 is a drawing for describing passive sound masking, according to embodiments of the present disclosure.

The passive sound masking is a sound masking technology in which a cocktail party effect for randomly playing a sound not familiar to the brain and ears is reflected. According to an embodiment of a sound masking algorithm of the sound masking technology, the sound masking algorithm may play random noise-based white noise in a sleep interval and may play pink noise for emphasizing a mid-low sound in a deep sleep interval.

The sound masking algorithm may randomly play the masking sound to implement a deep sleep therapy solution using the masking sound such as white noise or pink noise. For example, the sound masking algorithm may include a filter function for decreasing a change in sound and a random sound quality correction function.

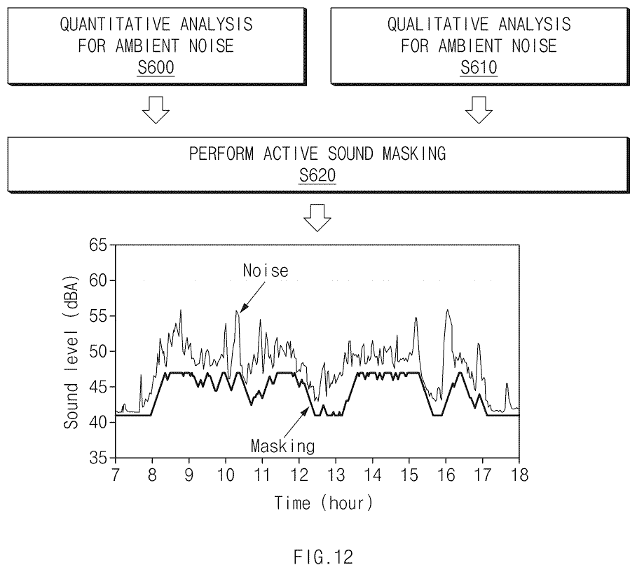

FIG. 12 is a drawing for describing active sound masking, according to embodiments of the present disclosure.

In an operation S600, the sound masking apparatus 100 of FIG. 1 may perform a quantitative analysis for ambient noise in a specific space (e.g., a vehicle, an office, a classroom, or a campground). For example, the sound masking apparatus 100 may analyze a quantitative numerical value (e.g., a noise stress index) for the ambient noise.

In an operation S610, the sound masking apparatus 100 may perform a qualitative analysis for the ambient noise. For example, the sound masking apparatus 100 may measure EEG which is a biometric signal and may analyze an EEG pattern.

In an operation S620, the sound masking apparatus 100 may perform active sound masking based on the result of performing the quantitative analysis for the ambient noise and/or the result of performing the qualitative analysis for the ambient noise.

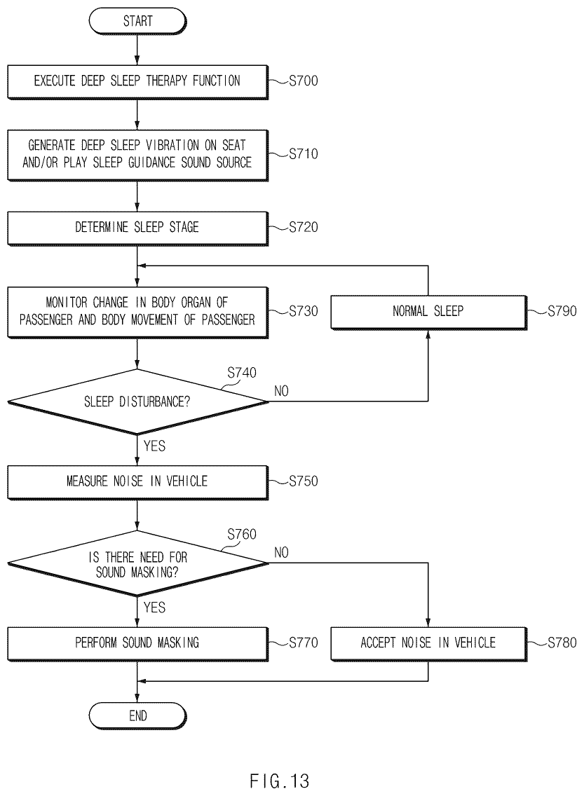

FIG. 13 is a flowchart illustrating a sound masking method, according to embodiments of the present disclosure.

In an operation S700, the controller 150 of the sound masking apparatus 100 of FIG. 1 may execute a deep sleep therapy function. The controller 150 may execute the deep sleep therapy function based on input data of a passenger, which may be received from a human interface device 110 of FIG. 1,

When the deep sleep therapy function is executed, the controller 150 may, in an operation S710, generate sleep vibration on the seat and/or may play a sleep guidance sound source. The controller 150 may control the seat controller 140 to generate vibration with a vibration frequency and/or a vibration pattern for guiding a passenger to have a deep sleep on the seat. Furthermore, the controller 150 may play the sleep guidance sound source using a sound source player and may output the sleep guidance sound source through a speaker.

In an operation S720, the controller 150 may determine a sleep stage by means of the sensing device 130 of FIG. 1. The controller 150 may measure a biometric signal using a biometric signal measurement device. The controller 150 may analyze a pattern of the measured biometric signal to determine a sleep stage. As an example, the controller 150 may measure an EEG signal using an EEG measurement device. The controller 150 may analyze a pattern of the measured EEG signal to determine a sleep stage. The sleep stage may be divided into a first sleep stage (or a sleep boundary stage), a second sleep stage (or a light sleep stage), and a third sleep stage (or a deep sleep stage).

In an operation S730, the controller 150 may monitor a change in body organ of the passenger and body movement of the passenger using the sensing device 130. The controller 150 may obtain image information by means of a camera. The controller 150 analyze the obtained image information to detect an eye blinking duration of the passenger, a body movement pattern of the passenger, or a combination thereof.

In an operation S740, the controller 150 may determine whether a sleep state is sleep disturbance based on the change in body organ of the passenger and the body movement of the passenger. When the eye blinking duration is less than or equal to a predetermined reference value (or reference time) (e.g., 400 ms) and/or when the body movement pattern is a REM sleep behavior disorder pattern, the controller 150 may determine that the sleep state is the sleep disturbance.

When it is determined that the sleep state is the sleep disturbance, the controller 150 may, in an operation S750, measure noise in a vehicle using a microphone.

In an operation S760, the controller 150 may determine whether there is a need for sound masking based on the measured noise. The controller 150 may compare a magnitude of the measured noise with a predetermined reference value. The controller 150 may determine that there is the need for the sound masking when the magnitude of the measured noise is greater than or equal to the predetermined reference value. When the magnitude of the measured noise is not greater than or equal to the predetermined reference value, the controller 150 may determine that there is no need for sound masking.

When it is determined that there is the need for the sound masking, the controller 150 may, in an operation S770, perform the sound masking. The controller 150 may determine a masking sound based on the sleep stage, the noise magnitude, REM sleep cycle information, emergency information, and/or the like. The controller 150 may automatically adjust a sound by means of a spatial acoustic noise analysis.

When it is determined that there is no need for sound masking, the controller 150 may, in an operation S780, determine that the noise in the vehicle is acceptable.

When the sleep state is not the sleep disturbance, the controller 150 may, in an operation S790, determine the sleep state of the passenger as normal sleep.

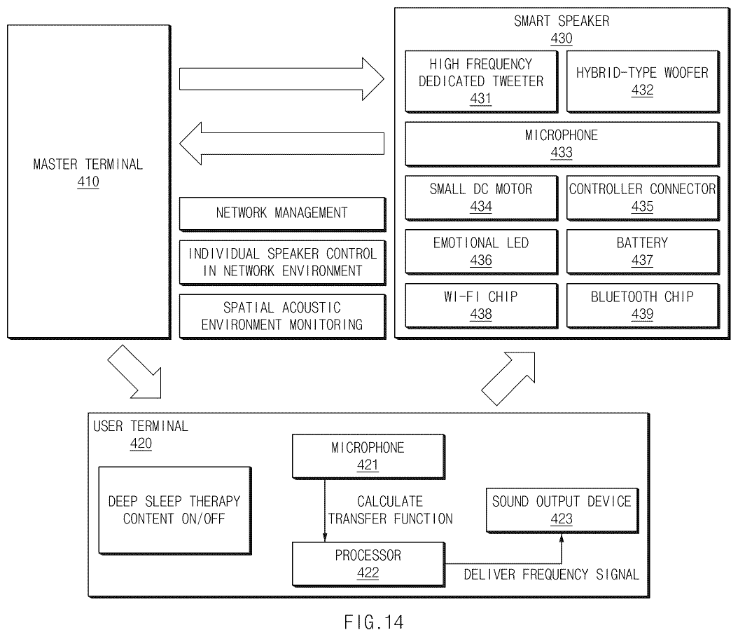

FIG. 14 is a block diagram illustrating a configuration of a sound masking system, according to embodiments of the present disclosure.

Referring to FIG. 14, a sound masking system 400 may include a master terminal 410, a user terminal 420, and a smart speaker 430, which are connected over a wired and/or wireless communication network. Each of the master terminal 410, the user terminal 420, and the smart speaker 430 may include a communication circuit, a processor, and a memory.

The master terminal 410 may be installed in a specific space (e.g., a vehicle, an office, or the like). The master terminal 410 may perform network management, individual speaker control in a network environment, spatial acoustic environment monitoring, or the like.

The master terminal 410 may analyze a sleep stage of a user using a biometric signal measurement device. The master terminal 410 may analyze a change in body organ of the user, a body movement pattern of the user, and/or the like using a camera installed in the specific space and may determine whether a sleep state is sleep disturbance.

When the sleep disturbance is recognized, the master terminal 410 may transmit a control command to execute deep sleep therapy content to the user terminal 420. The master terminal 410 may transmit sleep stage information of the user along with the control command.

The deep sleep therapy content may be installed in advance in the form of an application in the user terminal 420. The user terminal 420 may turn on or off the deep sleep therapy content based on the control command of the master terminal 410.

When the deep sleep therapy content is turned on, the user terminal 420 may measure noise (or ambient noise) in a spatial acoustic environment using a microphone 421. The user terminal 420 may select a masking sound source based on the sleep stage of the user and the measured noise. The user terminal 420 may transmit the selected masking sound source to the smart speaker 430.

The user terminal 420 may play and output the selected masking sound source through a sound output device 423. The sound output device 423 may include a function of recognizing whether the smart speaker 430 operates.

The smart speaker 430 may include a high frequency dedicated tweeter 431, a hybrid-type woofer 432, a microphone 433, a small DC motor 434, a controller connector 435, an emotional LED 436, a battery 437, a Wi-Fi chip 438, and a Bluetooth chip 439.

The smart speaker 430 may receive the masking sound source transmitted from the user terminal 420 through the Wi-Fi chip 438 and/or the Bluetooth chip 439. The smart speaker 430 may play and output the received masking sound source through the high frequency dedicated tweeter 431 and/or the hybrid-type woofer 432.

The smart speaker 430 may be a portable-type speaker, such as a cup holder-type smart speaker, a handle hanging-type smart speaker, or the like.

The smart speaker 430 may measure ambient noise using the embedded microphone 433. When the user experience (UX)-based smart speaker 430 is used, a peer-to-peer masking sound source may be played using the short-range microphone 433 near the user. Furthermore, a sound source obtained by synthesizing a natural sound with music may be used as a masking sound source to minimize the sense of difference.

As another example, the smart speaker 430 may analyze a type of the measured noise using an artificial intelligence (AI) server and may play a masking sound source optimized for the type of the noise. The smart speaker 430 may transmit the received noise to the AI server.

As another example, the smart speaker 430 may analyze user experience using the AI server and may play a masking sound source optimized for the user. The AI server may provide the masking sound source.

An omni-directional speaker may be embedded in the smart speaker 430 such that the smart speaker 430 maximizes a soundscape speaker use radius. Additionally, or alternatively, an echo canceller may be embedded in the smart speaker 430 such that the smart speaker 430 prevents a playback sound from being introduced into the microphone again.

As another example, the smart speaker 430 may analyze a magnitude and a frequency characteristic of noise measured by the microphone 433. The smart speaker 430 may select and play a masking sound source matched with the analyzed result among masking sound sources stored in a memory. The smart speaker 430 may adjust a level and timbre of the masking sound source in real time. A noise type analysis function and a user usability analysis function may be additionally applied to the smart speaker 430 in an active scheme.

Embodiments of the present disclosure may determine a sleep state of the user to guide the user to sleep and may mask a sleep disturbance sound, thus guiding the user to have a deep sleep.

Embodiments of the present disclosure may determine a sleep stage and sleep quality based on image information and/or biometric information and may play a masking sound based on the determined sleep stage and/or the determined sleep quality, thus guiding the user to sleep.

Hereinabove, although the present disclosure has been described with reference to example embodiments and the accompanying drawings, the present disclosure is not limited thereto. The embodiments of the present disclosure may be variously modified and altered by those having ordinary skill in the art to which the present disclosure pertains without departing from the spirit and scope of the present disclosure claimed in the following claims. Therefore, embodiments of the present disclosure are provided only for illustrative purposes and are not intended to limit the technical spirit of the present disclosure. The scope of the present disclosure should be construed on the basis of the accompanying claims, and all the technical ideas within the scope equivalent to the claims should be included in the scope of the present disclosure.

What is claimed is:

1. A sound masking apparatus, comprising:
a sensing device configured to measure image information and biometric information of a passenger in a vehicle; and
a controller connected with the sensing device, the controller configured to
determine a sleep state of the passenger based on the biometric information,
determine whether the passenger is disturbed in sleep in the sleep state of the passenger based on the image information,
measure noise in the vehicle in response to determining that the passenger is disturbed in sleep,
determine whether there is a need for sound masking based on the measured noise, and
perform the sound masking based on the sleep state in response to determining that there is the need for the sound masking.

2. The sound masking apparatus of claim 1, wherein the controller is configured to:
analyze a pattern of an electroencephalogram (EEG) signal measured by an EEG measurement device; and
determine the sleep state of the passenger based on the analyzed pattern of the EEG signal.

3. The sound masking apparatus of claim 1, wherein the controller is configured to:
monitor a change in a body organ of the passenger and body movement of the passenger by analyzing the image information; and
determine whether the passenger is disturbed in sleep based on at least one of the change in a body organ of the passenger or a body movement pattern, or any combination thereof.

4. The sound masking apparatus of claim 1, wherein the controller is configured to determine that the passenger is disturbed in sleep when at least one of an eye blinking duration of the passenger is less than or equal to a predetermined reference time or a body movement pattern is a rapid eye movement (REM) sleep behavior disorder pattern, or any combination thereof.

5. The sound masking apparatus of claim 1, wherein the controller is configured to:
compare a magnitude of the measured noise with a predetermined reference value; and
determine that there is the need for the sound masking when the magnitude of the measured noise is greater than or equal to the predetermined reference value.

6. The sound masking apparatus of claim 1, wherein the controller is configured to:
select white noise as a masking sound source when the sleep state switches from a sleep boundary stage to a sleep stage; and
play and output the selected white noise.

7. The sound masking apparatus of claim 6, wherein the controller is configured to randomly play the selected white noise.

8. The sound masking apparatus of claim 1, wherein the controller is configured to:
select pink noise as a masking sound source when the sleep state switches from a sleep stage to a deep sleep stage; and
play and output the selected pink noise.

9. The sound masking apparatus of claim 8, wherein the controller is configured to control a magnitude of the pink noise based on a magnitude of the measured noise.

10. The sound masking apparatus of claim 1, wherein the controller is configured to select and play a masking sound source based on a purpose of use of the sound masking.

11. A sound masking method, comprising:
determining a sleep state of a passenger in a vehicle based on biometric information of the passenger, the biometric information being measured by a sensing device;
determining whether the passenger is disturbed in sleep based on image information of the passenger, the image information being obtained by the sensing device in the sleep state of the passenger;
measuring noise in the vehicle in response to determining that the passenger is disturbed in sleep;
determining whether there is a need for sound masking based on the measured noise; and
performing the sound masking based on the sleep state in response to determining that there is the need for the sound masking.

12. The sound masking method of claim 11, wherein determining the sleep state of the passenger includes:
analyzing a pattern of an EEG signal measured by an EEG measurement device; and
determining the sleep state of the passenger based on the analyzed pattern of the EEG signal.

13. The sound masking method of claim 11, wherein determining whether the passenger is disturbed in sleep includes:
monitoring a change in a body organ of the passenger and body movement of the passenger by analyzing the image information; and
determining whether the passenger is disturbed in sleep based on at least one of the change in a body organ of the passenger or a body movement pattern, or any combination thereof.

14. The sound masking method of claim 11, wherein determining whether the passenger is disturbed in sleep includes:
determining that the passenger is disturbed in sleep when at least one of an eye blinking duration of the passenger is less than or equal to a predetermined reference time or a body movement pattern is a REM sleep behavior disorder pattern, or any combination thereof.

15. The sound masking method of claim 11, wherein determining whether there is the need for the sound masking includes:
comparing a magnitude of the measured noise with a predetermined reference value; and
determining that there is the need for the sound masking when the magnitude of the measured noise is greater than or equal to the predetermined reference value.

16. The sound masking method of claim 11, wherein performing the sound masking includes:
selecting white noise as a masking sound source when the sleep state switches from a sleep boundary stage to a sleep stage; and
playing and outputting the selected white noise.

17. The sound masking method of claim 16, wherein playing and outputting the selected white noise includes randomly playing the selected white noise.

18. The sound masking method of claim 11, wherein performing the sound masking includes:
selecting pink noise as a masking sound source when the sleep state switches from a sleep stage to a deep sleep stage; and
playing and outputting the selected pink noise.

19. The sound masking method of claim 18, wherein playing and outputting the selected pink noise includes controlling a magnitude of the pink noise based on a magnitude of the measured noise.

20. The sound masking method of claim 11, wherein performing the sound masking includes selecting and playing a masking sound source based on a purpose of use of the sound masking.
